(19) United States
(12) Patent Application Publication
(10) Pub. No.: US 2009/0281093 A1
(43) Pub. Date: Nov. 12, 2009

(54) ANTI-INFLAMMATORY COMPOUNDS

(75) Inventors: Anna Sparatore, Milan (IT); Piero Del Soldato, Monza (IT)

Correspondence Address: SCHNECK & SCHNECK, P.O. BOX 2-E, SAN JOSE, CA, 95109-0005, US

(73) Assignee: C.T.G. PHARMA S.R.L., Milan (IT)

(21) Appl. No.: 11/911,802

(22) PCT Filed: Jul. 29, 2005
(86) PCT No.: PCT/IB05/02401

§ 371 Date: Nov. 19, 2007

(30) Foreign Application Priority Data
Apr. 18, 2005 (IT) MI2005A000680

(51) Int. Cl. A61K 47/48; A61K 31/196; A61K 31/44; A61K 31/415; A61K 31/4025; A61K 31/5415; A61K 31/18; A61K 31/365; A61K 31/42; A61P 29/00

(52) U.S. Cl. 514/226.5; 514/567; 514/352; 514/406; 514/411; 514/605; 514/473; 514/378

(57) ABSTRACT

The present invention relates to novel non steroidal anti-inflammatory compounds (NSAIDs) that release hydrogen sulfide (H₂S). The present invention also provides methods for treating, preventing and/or reducing inflammation-associated diseases by releasing H₂S in the cardiovascular, connective tissue, pulmonary, gastrointestinal, respiratory, urogenital, nervous, or cutaneous systems as well as infective diseases employing said compounds.

### BACKGROUND

The anti-inflammatory, analgesic and antipyretic drugs are an heterogeneous group of compounds, often chemically unrelated, which nevertheless share certain therapeutic actions and side effects. They are frequently called non steroidal anti-inflammatory drugs or NSAIDs.

NSAIDs find their chief clinical application as anti-inflammatory agents in the treatment of muscle-skeletal disorders, such as rheumatoid arthritis, osteoarthritis, and ankylosing spondylitis. In general, NSAIDs provide only symptomatic relief from the pain and inflammation associated with the disease and do not arrest the progression of the pathological injury to tissue.

Although NSAIDs had been known to inhibit a wide variety of reactions in vitro, the first convincing relationship was established by Vane et al. in 1971 when they demonstrated that low doses of aspirin and indomethacin inhibited the enzymatic production of prostaglandins. The first enzyme in the prostaglandin synthetic pathway is prostaglandin endoperoxide synthase, or fatty acid cyclooxygenase. It is now appreciated that there are two forms of cyclooxygenase termed cyclooxygenase-1 (COX-1) and cyclooxygenase-2 (COX-2).

More specifically, it is commonly recognized that the beneficial actions of NSAIDs can be associated with inhibition of COX-2 whereas their harmful side effects are associated with inhibition of COX-1.

In addition to sharing many therapeutic activities, NSAIDs share several unwanted side effects. The most common is a propensity to induce gastric or intestinal ulceration that can sometimes be accompanied by anemia from the resultant blood loss. Patients who use NSAIDs in a chronic basis have about three times greater relative risk for serious adverse gastrointestinal events compared to non users (S. E. Gabriel, et al. Risk for serious gastrointestinal complications related to use of non steroidal antiinflammatory drugs Ann. Inter. Med. 1991, 115, 787-796).

More recently, it has been shown that compounds that selectively inhibit COX-2 with only very weak activity on COX-1 such as rofecoxib, for example, are responsible of serious cardiovascular problems after long term administration.

### FIELD OF THE INVENTION

This invention relates to the field of new antiiflammatory compounds that release hydrogen sulfide that possess improved gastric tolerability.

Hydrogen sulfide (H₂S) has been traditionally viewed as a toxic gas. It is also, however, endogenously generated from cysteine metabolism.

Cysteine is the natural supplier of hydrogen sulfide and the two H₂S producing enzymes actually known are cystathionine β-synthase (CBS) in the brain and cystathionine γ-lyase (CSE) in the smooth muscle. In Journal of Neurophysiology, vol. 70, No. 1, July 1993, the actions of hydrogen sulfide on dorsal raphe serotonergic neurons in vitro is described. In Molecular Neurobiology, vol. 26, pp 6-12, 2002, studies are reported on H₂S relaxation of smooth muscle in synergy with nitric oxide (NO) and on its use as neuromodulator. From EMBO Journal, vol. 21, No. 2, 6008-6016, 2001, the vasorelaxant effect of H₂S as a novel endogenous gaseous K_{ATP} channel opener is known.

Tissue damage by infection, trauma, toxins, abnormally low or high temperatures and other reasons, usually leads to formation of increased amounts of pathogenetic mediators, such as prostaglandins, cytokines, leukotrienes, interleukins, oxy-, thiyl- and nitrogen-free radicals, interacting each other. The research has been focused in the past to develop effective therapeutic agents able to counteract the deleterious effects of such mediators, but results have been so far either unsatisfactory or only partially satisfactory. An alternative and/or complementary approach is to develop agents able to potentiate the natural defences. This can be done also through the contribution of a mediator, such as, CO, NO that can cooperate to potentiate the natural tissue defences.

Therefore, the ideal approach is to develop compounds able to both counteract aggressive and pathogenetic factors and potentiate defensive substances. This can be surprisingly obtained by combining chemically a drug known to counteract one or more of the above mentioned aggressive factors with a moiety able to release directly or indirectly the defensive substance (hydrogen sulfide). The results have been surprising, in that not only the safety was dramatically improved but also the efficacy was sometimes increased.

### DESCRIPTION OF THE INVENTION

Object of the present invention are compounds of general formula:

D-X—Y—S (I)

wherein D is a non steroidal anti-inflammatory drug having a IC₈₀ (μM) of COX 2≦than IC₈₀ (μM) of COX 1; X is zero, C═O, COOR or CONH, R being straight or branched C₁-C₄-alkyl;
Y is zero, —O— (CH₂)ₙ—O— or —OOC—(CH₂)ₙ—COO—, where n is 2-10;
S is a moiety capable per se or in combination with the drug to release H₂S;
pharmaceutical composition containing them as well their use for treating, preventing or reducing inflammation associated with cardiovascular, respiratory, connective tissue, nervous, gastrointestinal, cutaneous, infective, and urogenital diseases.

The moiety (D) present in the new compounds object of the present is a NSAID that release H₂S and where the parent compound exhibits a IC₈₀ (μM) of COX 2 that is ≦than IC₈₀ (μM) of COX 1.

For the determination of the IC₈₀ (μM) we refer to the human whole blood (WBA) assay described in the paper of T. D. Warner et al. Nonsteroid drug selectivities for cyclo-oxygenase-1 rather than cyclo-oxygenase-2 are associated with human gastrointestinal toxicity: A full in vitro analysis. Proc. Natl. Acad. Sci. 96, 7563-7569, 1999.

The present invention is based on the discovery that it is possible to link H₂S releasing moieties to a pharmacologically active compound helpful for treating disorders in the cardiovascular, connective tissue, pulmonary, gastrointestinal, respiratory, urogenital, nervous, or cutaneous systems and infective diseases. The resulting compounds have good bioavailability, increased safety and maintain good efficacy.

The parent drugs (i.e. the drugs in which the modification with H₂S releasing moiety can be applied) in the present invention can be selected within the class of NSAIDs compounds, that parent compound posses a IC₈₀ (μM) of COX 2≦than IC₈₀ (μM) of COX 1 (according to the reference mentioned above) such as diclofenac, flufenamate, niflumic acid, celecoxib, etodolac, meloxicam, nimesulide, rofecoxib, valdecoxib, parecoxib, lumiracoxib diflunisal etc.

Also others non steroidal anti-inflammatory parent compounds, that possess a IC₈₀ (μM) of COX 2≦than IC₈₀ (μM) of COX 1 (defined as above), and compounds that are nitric oxide donors derivatives of the above mentioned compounds, are considered part of the present invention.

When the compounds include at least one asymmetric carbon atom, the products can be used in racemic mixture or in form of single enantiomer.

In the present invention the parent compound is considered in its original form or in a proper modification to allow the chemical manipulation with H₂S releasing moieties.

Also parent drugs able to release nitric oxide exogenously or endogenously are useful for the present invention.

Other substances releasing or stimulating the release of hydrogen sulfide that can be linked to drugs are N-acetyl-penicillamine, S-allyl-cysteine, bucillamine, carbocysteine, cysteamine, cystathionine, homocysteine, mecysteine, methionine, pantetheine, penicillamine, penicillamine disulfide, thioacetic acid, thiodiglycolic acid, thioglycolic acid, thiolactic acid, 2-thiolhistidine, thiomalic acid, thiosalicylic acid, tiopronin.

Other substances releasing and/or stimulating the release of hydrogen sulfide that can be linked to drugs are 5-(p-hydroxyphenyl)-3H-1,2-dithiol-3-thione, 1,3-dithiol-2-thione-5-carboxylic acid, 3-thioxo-3H-1,2-dithiole-5-carboxylic acid, 3-thioxo-3H-1,2-dithiole-4-carboxylic acid.

Further groups releasing H₂S also in combination with groups that release also nitric oxide or carbon oxide are part of the present invention.

The substances can be linked via different linking groups such as esters, amides, imides, sulfonamides, azo groups, carbamates, carbonates, anhydrides, acetals, thioacetals, etc.

Bifunctional linkers known to the expert in the field (such as ethyl, propyl, or butyl diols; di-amines; hydroxy amines, etc.) can be optionally present when they are necessary to link the drug to the substances releasing and/or stimulating the release of hydrogen sulfide.

Also salts pharmaceutically acceptable that directly or indirectly are capable to release H₂S are part of the present invention.

The compounds of the present invention can be administered in the form of any pharmaceutical formulation, the nature of which will depend upon the route of administration and the nature of the disease to be treated. These pharmaceutical compositions can be prepared by conventional methods, using compatible, pharmaceutically acceptable excipients or vehicles. Examples of such compositions include capsules, tablets, syrups, powders and granulates for the preparation of extemporaneous solutions, injectable preparations, rectal, nasal, ocular, vaginal etc. A preferred route of administration is the oral route.

The following non-limitative examples further describe and enable an ordinary skilled in the art to make and use the invention.

### Example 1

### Synthesis of 2-[(2,6-dichlorophenyl)amino]benzeneacetic acid 4-(3H-1,2,dithiole-3-thione-5-yl)-phenyl ester

To 280 mmol of sulfur, 40 mmol of anethole were added. After heating at 200° C. for 6 hours, 2.5 g of anethole trithione (ADT) were obtained. The product, washed with ether, was crystallized by ethyl acetate: melting point 110-111° C. Then 1.5 g of ADT were mixed with 7.5 g of pyridine HCl and the mixture was heated for 25 minutes at 215° C. After cooling, 1N HCl in excess was added and the precipitate was filtered, washed and crystallized from ethanol. The obtained 5-(p-hydroxyphenyl)-3H-1,2-dithiole-3-thione melted at 191-192° C.

Diclofenac free acid (766 mg, 2.59 mmol) was dissolved in dichloromethane (90 ml) and 5-(p-hydroxyphenyl)-3H-1,2-dithiol-3-thione (585 mg, 2.59 mmol), dicyclohexylcarbodiimide (DCC) (587 mg, 2.85 mmol) and a catalytic amount (13 mg) of 4-dimethylaminopyridine (DMAP) were added. The mixture was stirred at room temperature for 40 minutes.

At the end of the reaction the mixture was filtered to remove the dicyclohexylurea (DCU), and the solution was washed with 0.1 N NaOH and cold water. The organic solution was dried on sodium sulphate and evaporated. The residue was chromatographed on silica gel column eluting with a mixture of dichloromethane/cyclohexane 8/2. The obtained compound had a melting point of 85-88° C.

### Example 2

### Synthesis of 2′,4′-difluoro-4-hydroxy-[1,1′-biphenyl]-3-carboxylic acid 4-(3H-1,2,dithiole-3-thione-5-yl)-phenyl ester

A 1N solution of dicyclohexylcarbodiimide (DCC) in dichloromethane (455 mg, 2.2 mmol) was added to 100 ml of a dichloromethane solution containing of 5-(p-hydroxyphenyl)-3H-1,2-dithiol-3-thione (451.2 mg, 1.99 mmol), prepared as described in example 1, diflunisal (500 mg, 1.99 mmol) and 4-dimethylaminopyridine (DMAP) (245 mg, 1.99 mmol).

The mixture was stirred at room temperature under nitrogen for 20 hours. At the end of the reaction dicyclohexylurea (DCU) was removed by filtration.

The solution was washed with 0.1N NaOH and cold water. The organic solution was then dried on anhydrous sodium sulphate and evaporated. After removal of the solvent, the mixture was chromatographed on silica gel eluting with a mixture of dichloromethane/methanol (99.5/0.5).

The compound after washing first with ether, then with ethanol and crystallization with ethyl acetate had a melting point of 202-204° C.

### Example 3

Operating as described in examples 1 and 2, but employing lumeracoxib, flufenamic acid, niflumic acid instead of diflunisal the corresponding 4-(3H-1,2,dithiole-3-thione-5-yl)-phenyl ester are obtained.

### Example 4

### Activity and Safety

Thirty male rats (150-200 g) were divided into three groups of 10 animals/group and treated with vehicle, diclofenac (20 mg/kg) and the compound of example 1 (36 mg/kg) for five days once daily by gavage. The vehicle consisted in 1 mL of acidified aqueous solution. At the fifth day, survival animals were sacrificed 6 hr after the treatment, by an overdose of ether, and autopsied to detect any pathological changes, particularly in the gastrointestinal tract. Prostaglandin E2 was evaluated in the liver homogenate by standard enzyme immunoassay procedure as described by Warner T D et al. (J.P.E.T. 2004, 310, 642-7). The results of this experiment are shown in the following Table 2:

| | | % gastrointestinal |
|---|---|---|
| | % deaths | ulcer |
| Control | 0 | 0 |
| Diclofenac | 70 | 100 |
| Compound example 1 | 0 | 0 |

Prostanoid formation in liver homogenates of animals treated with compound example 1 or diclofenac was always markedly reduced (by 90% or more) as compared to the samples from vehicle-treated animals. On the whole these results indicate that Compound example 1 is significantly better tolerated than diclofenac while maintaining fully the activity (i.e. the ability to inhibit prostaglandin formation).

1. Compounds of general formula
D-X—Y—S (I)
wherein D is a non steroidal anti-inflammatory drug having a IC₈₀ (μM) of COX 2≦than IC₈₀ (μM) of COX 1; X is zero, C═O, COOR or CONH, R being straight or branched C₁-C₄-alkyl;
Y is zero, —O—(CH₂)ₙ—O— or —OOC—(CH₂)ₙ—COO—, where n is 2-10;
S is a moiety capable per se or in combination with the drug to release H₂S.

2. Compounds according to claim 1, characterized in that the drug (D) is selected among diclofenac, flufenamate, niflumic acid, celecoxib, etodolac, meloxicam, nimesulide, rofecoxib, valdecoxib, parecoxib, lumiracoxib, diflunisal.

3. Compounds according to claim 1, characterized in that the moiety S is selected from the group consisting of N-acetyl-penicillamine, S-allyl-cysteine, bucillamine, carbocysteine, cysteamine, cystathionine, homocysteine, mecysteine, methionine, pantetheine, penicillamine, penicillamine disulfide, thioacetic acid, thiodiglycolic acid, thioglycolic acid, thiolactic acid, 2-thiolhistidine, thiomalic acid, thiosalicylic acid, tiopronin, 5-(p-hydroxyphenyl)-3H-1,2-dithiol-3-thione, 1,3-dithiol-2-thione-5-carboxylic acid, 3-thioxo-3H-1,2-dithiole-5-carboxylic acid, 3-thioxo-3H-1,2-dithiole-4carboxylic acid.

4. Compounds according to claim 1, characterized in that the moiety S is selected from groups releasing H₂S also in combination with groups that release nitric oxide or carbon oxide.

5. Compounds according to claim 1 for treating, preventing or reducing inflammation associated with cardiovascular, respiratory, connective tissue, nervous, gastrointestinal, cutaneous, infective, urogenital diseases.

6. Pharmaceutical composition for treating, preventing or reducing inflammation associated with cardiovascular, respiratory, connective tissue, nervous, gastrointestinal, cutaneous, infective, urogenital diseases, comprising a compound of formula (I) as active ingredient and a pharmaceutically acceptable adjuvant or carrier.
